(19) United States
(12) Patent Application Publication
(10) Pub. No.: US 2023/0310139 A1
(43) Pub. Date: Oct. 5, 2023

(54) IMPLANTABLE MEDICAL DEVICE WITH VISUAL INDICATORS

(71) Applicant: BOSTON SCIENTIFIC SCIMED, INC., Maple Grove, MN (US)

(72) Inventors: John O’Driscoll, Galway (IE); Daniel Tuck, Galway (IE); Darren Gerard Curran, Galway (IE); Fionn Stapleton, Galway (IE)

(73) Assignee: BOSTON SCIENTIFIC SCIMED, INC., (omitted)

(21) Appl. No.: 18/193,431

(22) Filed: Mar. 30, 2023

Related U.S. Application Data
(60) Provisional application No. 63/325,772, filed on Mar. 31, 2022.

(51) Int. Cl. A61F 2/04

(52) U.S. Cl. CPC A61F 2/04; A61F 2002/045; A61F 2250/0098

(57) ABSTRACT

An implantable medical device includes an elongate body extending from a distal region to a proximal region, at least one of the distal region and the proximal region including a flared region defining an inner flared surface. One or more visual indicators are positioned relative to the inner flared surface such that the one or more visual indicators are visible endoscopically from a position within or proximate the implantable stent.

### CROSS-REFERENCE TO RELATED APPLICATIONS

This application claims the benefit of U.S. Pat. Application Serial No. 63/325,772, filed Mar. 31, 2022, which is incorporated herein by reference.

### TECHNICAL FIELD

The present disclosure pertains to implantable medical devices. More particularly, the disclosure is directed to implantable medical devices that include visual indicators showing a relative orientation of the implantable medical device once implanted.

### BACKGROUND

A variety of implantable medical devices are implanted within the human body for a variety of different purposes. Some implantable medical devices are agnostic as to the relative position in which the implantable medical device is implanted within an internal body structure. Internal body structures may include an interior of an organ, for example, or a portion of a body lumen, such as the esophagus, trachea, colon, biliary duct, vasculature, etc. In some cases, the implantable medical device may be symmetrical left to right, or top to bottom, or front to back. In some cases, however, there may be a desire to know the relative orientation of the implantable medical device. For example, the relative rotational orientation of the implantable medical device about its axis relative to the internal body structure may be of interest. For instance, indication of the relative orientation of the implantable medical device relative the sagittal plane, the coronal plane, and/or the transverse plane through the human body may be desired. In some instances, indication of the orientation of the implantable medical device relative to a vertically upward position (i.e., a superior orientation), or which part of the implantable medical device is oriented at a vertically downward position (i.e., an inferior orientation), or perhaps an intermediate position relative to vertical may be desired. A need remains for implantable medical devices that include a visually recognizable way to determine the relative orientation of the implantable medical device once implanted.

### SUMMARY

This disclosure provides design, material, manufacturing method, and use alternatives for medical devices. As an example, an implantable stent includes an elongate body extending from a distal region to a proximal region, the elongate body including one or more filaments that are woven together to form the elongate body, the elongate body defining an outer stent surface and an inner stent surface. One or more visual indicators are positioned relative to the inner stent surface such that the one or more visual indicators are visible endoscopically from a position within or proximate the implantable stent.

Alternatively or additionally, the distal region or the proximal region may include a flared end, the inner stent surface extending into the flared end, and the one or more visual indicators are positioned relative to the inner stent surface within the flared end.

Alternatively or additionally, the one or more visual indicators may include two or more visual indicators that are circumferentially equidistantly spaced apart on the inner stent surface, each of the two or more visual indicators being visually distinguishable from the other position indicators.

Alternatively or additionally, at least some of the two or more visual indicators may include objects that are secured relative to the inner stent surface.

Alternatively or additionally, the implantable stent may further include a polymeric coating over at least part of the elongate body.

Alternatively or additionally, at least some of the two or more visual indicators may include color changes introduced into the polymeric coating.

Alternatively or additionally, at least one of the one or more visual indicators may include a radiopaque material.

As another example, an implantable medical device is adapted to be deployed endoscopically. The implantable medical device includes an elongate body extending from a distal region to a proximal region, at least one of the distal region and the proximal region including a flared region defining an inner flared surface. Two or more visual indicators are secured relative to the inner flared surface, each of the two or more visual indicators positioned to be visible endoscopically.

Alternatively or additionally, each of the two or more visual indicators may be visually distinguishable from others of the two or more visual indicators.

Alternatively or additionally, each of the two or more visual indicators may include a color different from a color of any other of the two or more visual indicators.

Alternatively or additionally, the implantable medical device may include three visual indicators that are equidistantly spaced apart on the inner flared surface.

Alternatively or additionally, the implantable medical device may include four visual indicators that are equidistantly spaced apart on the inner flared surface.

Alternatively or additionally, at least some of the two or more visual indicators may include objects that are secured relative to the inner flared surface.

Alternatively or additionally, the implantable medical device may further include a polymeric coating over at least part of the elongate body.

Alternatively or additionally, at least some of the two or more visual indicators may include color changes introduced into the polymeric coating.

Alternatively or additionally, at least one of the two or more visual indicators may include a radiopaque material.

As another example, an implantable stent includes an elongate body extending from a distal region to a proximal region, the elongate body including one or more filaments that are woven together to form the elongate body, the elongate body moveable between a collapsed configuration for delivery and an expanded configuration for deployment, the elongate body including a constant diameter main segment and a flared end segment, the flared end segment including an inner flared surface and an outer flared surface. A plurality of visual indicators are secured relative to the inner flared surface such that each of the plurality of visual indicators are visible endoscopically.

Alternatively or additionally, the plurality of visual indicators may include three visual indicators that are equidistantly spaced apart on the inner stent surface, each of the three visual indicators a unique color relative to the other two visual indicators.

Alternatively or additionally, the plurality of visual indicators may include four visual indicators that are equidistantly spaced apart on the inner stent surface, each of the four visual indicators a unique color relative to the other three visual indicators.

Alternatively or additionally, at least some of the plurality of visual indicators may include a pigment added to a portion of a polymeric coating disposed over the elongate body.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic view of an illustrative implantable medical device implanted within a pyloric sphincter of a patient’s stomach along with an endoscopically deployed visualization device providing visualization of the illustrative implantable medical device;

FIG. 2 is a perspective view of the illustrative implantable medical device of FIG. 1;

FIG. 3 is an end view of the illustrative implantable medical device of FIG. 1, including a total of four visual indicators;

FIG. 4 is a side view of an illustrative implantable medical device;

FIG. 4A is an enlarged view of a portion of the illustrative implantable medical device of FIG. 4;

FIG. 5 is a cross-sectional view taken along line 5-5 of FIG. 4;

FIG. 6 is a schematic end view of the illustrative implantable medical device, including a total of four visual indicators;

FIG. 7 is a schematic end view of the illustrative implantable medical device, including a total of three visual indicators;

FIG. 8 is a schematic end view of the illustrative implantable medical device, including a total of four visual indicators; and

FIG. 9 is a schematic end view of the illustrative implantable medical device, including a total of three visual indicators.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

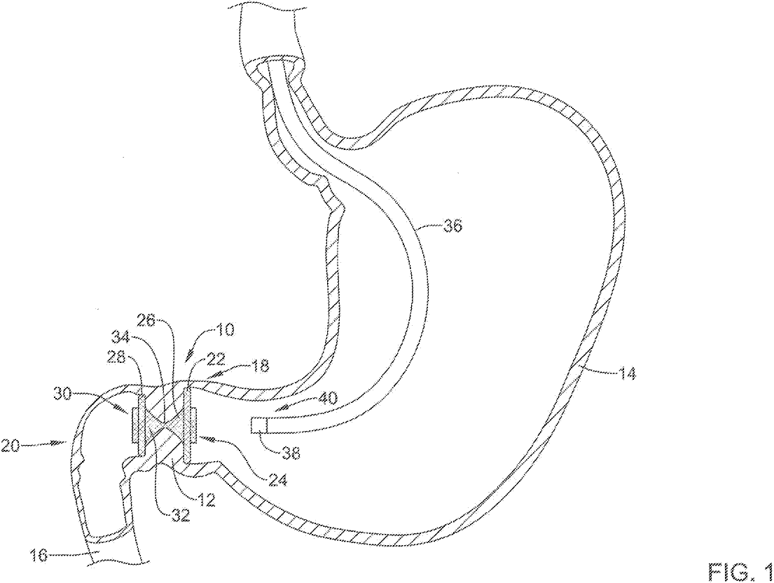

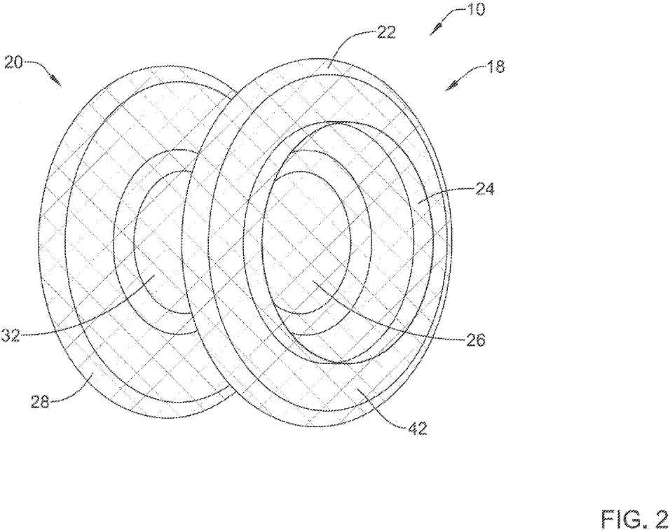

FIG. 1 is a schematic view of an illustrative implantable medical device 10, shown as a stent, that has been implanted within a pyloric sphincter 12 of a patient’s stomach 14. FIG. 2 is a perspective view of the implantable medical device 10. The stomach 14 is shown partially cut away in order to visualize the implantable medical device 10 and the pyloric sphincter 12. It will be appreciated that the pyloric sphincter 12 functions as a one-way valve, allowing partially digested food from the patient’s stomach 14 to pass into their duodenum 16 to be further digested. In some instances, the pyloric sphincter 12 itself may not be adequate to perform this function, or at least to sufficiently perform this function. A variety of medical conditions may contribute to a loss of this function, for example. Other medical conditions may be worsened or even caused by the pyloric sphincter 12 losing function.

Accordingly, the patient’s medical situation may suggest implantation of the implantable medical device 10. In this regard, the implantable medical device 10 may have an overall shape that facilitates implantation into the pyloric sphincter 12, including a proximal region 18 and a distal region 20. The proximal region 18 includes an anchoring flange or ring 22, an inflow region 24 and a tapered region 26. The distal region 20 includes an anchoring flange or ring 28, an outflow region 30 and a tapered region 32. An intermediate region 34 joins the tapered region 26 of the proximal region 18 to the tapered region 32 of the distal region 20. It will be appreciated that partially digested food moving through the stomach 14 will enter the inflow region 24 and be guided into and through the tapered region 26, through the intermediate region 34, into and through the tapered region 32 and out the outflow region 30. As a result, the implantable medical device 10 is configured to limit a rate at which partially digested food exits the stomach 14 into the small intestine 16, and can limit retrograde flow from the small intestine 16 into the stomach 14.

It will be appreciated that the anchoring ring 22 is configured to fit against a proximal portion of the pyloric sphincter 12 while the anchoring ring 28 is configured to fit against a distal portion of the pyloric sphincter 12. Together, the anchoring ring 22 and the anchoring ring 28, together with the tapered region 26 and the tapered region 32 serve both to accommodate the natural anatomy of the pyloric sphincter 12 and to anchor the implantable medical device 10 relative to the pyloric sphincter 12. The anchoring ring 22 and the anchoring ring 28 may be dimensioned to help hold the implantable medical device 10 in place, resisting the natural peristaltic movement of the stomach 14.

In some instances, as shown, the implantable medical device 10 may include a tubular scaffold formed of a braided structure that is formed by braiding together one or more, or a plurality of filaments. The braided structure may be formed by braiding together one or several filaments that extend in a first helical direction and one or several filaments that extend in a second helical direction different from the first helical direction. Each of the filaments extending in the first helical direction may extend continuously through the inflow region 24, the anchoring flange or ring 22, and the tapered region 26 of the proximal region 18, as well as the tapered region 32, the anchoring flange or ring 28, and the outflow region 30 of the distal region 20. Likewise, each of the filaments extending in the second helical direction may extend continuously through the inflow region 24, the anchoring flange or ring 22, and the tapered region 26 of the proximal region 18, as well as the tapered region 32, the anchoring flange or ring 28, and the outflow region 30 of the distal region 20. The tubular scaffold may include cells or interstices formed between adjacent filaments extending generally parallel in the first helical direction which intersect with adjacent filaments extending generally parallel in the second helical direction. At least some of the filaments may be metallic filaments. At least some of the filaments may be polymeric filaments, for example. The implantable medical device 10 may include a polymeric covering or coating that is disposed over the braided structure, for example. The individual filaments may be encapsulated within the polymeric covering or coating, particularly if the polymeric covering or coating is applied by dip coating.

In some cases, the implantable medical device 10 may not be used to replace the functionality of the pyloric sphincter 12, but instead may be used to occlude flow of materials through a body passage such as the pyloric sphincter 12. For example, treatment methods for various medical conditions, such as obesity, diabetes, or duodenal ulcers, can involve bypassing the duodenum or restricting flow of materials through the duodenum. In some cases, the implantable medical device 10 may be used to inhibit or block passage of materials (fluid, chyme, etc.) from the stomach 14 through the pyloric sphincter 12 and into the duodenum 16. Additional details regarding possible uses of the implantable medical device 10 and the construction thereof may be found for example in U.S. Pat. Application Serial No. 63/307,764 filed Feb. 8, 2022, entitled DEVICES, SYSTEMS, AND METHODS FOR OCCLUDING AN ANATOMICAL PASSAGE, and U.S. Pat. Application Serial No. 63/307,771 filed Feb. 8, 2022, entitled DEVICES, SYSTEMS, AND METHODS RESISTING MIGRATION AND METHOD OF IMPLANTATION THEREOF, which applications are incorporated by reference herein.

As seen in FIG. 1, an endoscope 36 including a visualization tool 38 disposed at a distal end 40 of the endoscope 36 has been extended through the esophagus into the stomach 14 and is proximate the implantable medical device 10. Accordingly, the visualization tool 38, which may represent a lens or camera, for example, or the terminal end of a fiber optic cable that is able to carry light back to a sensor or camera, is able to visualize a portion of the implantable medical device 10 after the implantable medical device 10 has been implanted. Once the implantable medical device 10 has been implanted, it will be appreciated that only portions of the implantable medical device 10 will be visible endoscopically, as outer portions of the implantable medical device 10 that are facing and/or in contact with tissue will be obstructed by tissue and thus cannot easily be visualized. In some cases, the visualization tool 38 is able to see an interior or inner surface, or an end surface, such as a surface 42 (with reference to FIG. 2) within the proximal region 18 of the implantable medical device 10. The visualization tool 38 may also be able to see a portion of the inflow region 24 and an interior of the tapered region 26. Accordingly, it is possible to visually ascertain an orientation, such as but not limited to, a rotational orientation of the implantable medical device 10 with respect to the patient’s anatomy.

In order to make it easier to ascertain the relative rotational orientation of the implantable medical device 10 once it has been implanted (such as but not limited to implantation within the pyloric sphincter 12), the implantable medical device 10 includes visual indicators that are disposed relative to the proximal region 18 of the implantable medical device 10. The implantable medical device 10 may include one, two, three, four or more visual indicators that are each visible to a viewing device such as the visualization tool 38 of the endoscope 36 that is positioned either proximal of the implantable medical device 10, where the visualization tool 38 is able to see an end view of the implantable medical device 10 including end surfaces and/or interior surfaces visible from the open end of the implantable medical device 10, or extending through a lumen within the implantable medical device 10, where the visualization tool 38 of the endoscope 36 is able to see an interior surface of the implantable medical device 10. For example, the implantable medical device 10 may include one visual indicator such that viewing the proximal region 18 allows visualization of the one visual indicator relative to the rest of the implantable medical device 10. This allows a determination that the one visual indicator is at or near top dead center (i.e., the 12 o′clock position), for example, relative to the rest of the proximal region 18.

Having two or more visual indicators, particularly if each of the two or more visual indicators are visually distinctive, can aid in determining relative orientation of the implantable medical device 10, particularly if the two or more visual indicators are visible but the rest of the implantable medical device 10 is not easily seen. If for example, two visual indicators are disposed along opposite sides of the surface 42, 180 degrees apart defining a line bisecting the surface 42, it is possible to visually ascertain the relative rotational orientation of the implantable medical device 10 by locating the two visual indicators and determining their relative rotational positions with the visualization tool 38 of the endoscope 36. For example, one visual indicator may have a relative position at 70 degrees (relative to a particular frame of reference provided by the visualization tool 38) while the other visual indicator may have a relative position at 160 degrees relative to that same frame of reference. By knowing where the two visual indicators are secured relative to the implantable medical device 10, the relative orientation of the implantable medical device 10 relative to the body structure in which it is implanted can be ascertained with the visualization tool 38 of the endoscope 36.

Regardless of how many visual indicators are included, in some instances each of the visual indicators (if more than one) are visually distinguishable from each other. In some cases, each visual indicator is a different color. In some cases, each visual indicator is a different size. In some cases, each visual indicator is a different shape. Combinations of different colors, different sizes and/or different shape are also contemplated. For instance, it will be appreciated that a set of two or more visual indicators may include multiple distinguishing features. For example, a first visual indicator may be round and yellow while another visual indicator may be square and red. A first visual indicator may be small and diamond-shaped. A second visual indicator may be large and diamond-shaped. These are just examples. The visual indicators may be formed in place on the implantable medical device 10. In some instances, the visual indicators may be separately formed and subsequently secured in place on the implantable medical device 10.

In some instances, one or more of the visual indicators may be formed from a radiopaque material and then secured to the implantable medical device 10. Having at least one of the visual indicators formed of a radiopaque material may aid in locating the position of the implantable medical device 10 during non-visible imaging modalities. Examples of radiopaque materials include but are not limited to barium sulfate, bismuth compounds and tungsten.

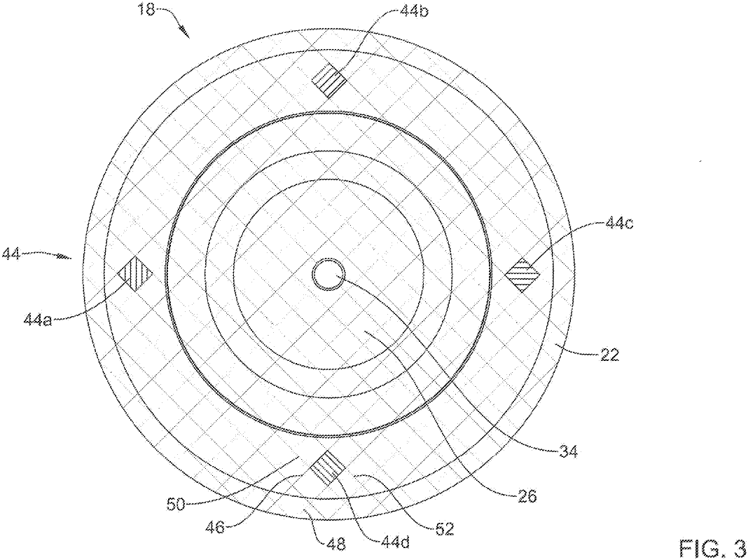

FIG. 3 is an end view of the proximal region 18 of the implantable medical device 10. As seen in FIG. 3, the implantable medical device 10 includes a total of four visual indicators 44, individually labeled as 44a, 44b, 44c and 44d. As shown, each of the four visual indicators 44 occupies one cell of the braided structure forming the implantable medical device 10. Looking for example at the visual indicator 44d, the visual indicator 44d occupies a cell formed by the intersections of filaments 46, 48, 50 and 52. In some cases, at least some of the visual indicators 44 may be larger than one cell. For instance, in some instances each of the visual indicators 44 may occupy four cells or interstices of the stent scaffold in a 2 × 2 grid. In some cases, each of the visual indicators 44 may be a different size in order to make it easier to visually distinguish between a particular visual indicator 44 and the other visual indicators 44.

In some cases, each of the visual indicators 44 may be a unique color in order to more easily distinguish between the visual indicators 44. Some unique colors that could be used include, black, white, red, blue, green, yellow, orange, purple, etc. As an illustrative but non-limiting example, say that the visual indicator 44a is white, the visual indicator 44b is blue, the visual indicator 44c is yellow and the visual indicator 44d is red. As another example, say that the visual indicator 44a is green, the visual indicator 44b is orange, the visual indicator 44c is purple and the visual indicator 44d is yellow. Depending on the light wavelengths at which the visualization tool 38 is most receptive to, other colors may be used as well.

The visual indicators 44 may be formed in any fashion. As an example, each of the visual indicators 44 may be formed by adding a particular pigment or other source of color to the polymeric coating or covering extending over at least the proximal region 18 of the implantable medical device 10. For example, a particular pigment may be added to a small amount of the polymer that forms the polymeric coating or covering, and can be mixed together and then coated onto the polymeric coating or covering. In some cases, at least some of the visual indicators 44 may be separately formed and subsequently secured in place to the surface 42. For example, colored discs or other shapes may be adhesively secured to the surface 42. If the colored discs are metallic, they may be welded or soldered into position on the surface 42, such as by welding or soldering the colored discs to one or more of the metallic filaments forming the tubular scaffold of the stent. The visual indicators 44 may be considered as being equidistantly spaced circumferentially around the central longitudinal axis of the implantable device 10.

In some instances, each visual indicator 44 may be a unique color, a unique shape and/or a unique size relative to the other visual indicators 44. As an illustrative but non-limiting example, the visual indicator 44a may be a circle, the visual indicator 44b may be a square, the visual indicator 44c may be a triangle, and the visual indicator 44d may be a polygon having at least five sides. As another illustrative but non-limiting example, the visual indicator 44a may be a circle having a diameter of 1 millimeter (mm), the visual indicator 44b may be a circle having a diameter of 2 mm, the visual indicator 44c may be a circle having a diameter of 3 mm and the visual indicator 44d may be a circle having a diameter of 4 mm. These are just examples.

While FIG. 3 shows a total of four of the visual indicators 44, each circumferentially spaced about 90 degrees apart around the central longitudinal axis of the implantable device 10, this is not required in all cases. For example, there may be a total of three of the visual indicators 44, each circumferentially spaced about 120 degrees apart. In some cases, there may be a total of two of the visual indicators 44, each spaced about 180 degrees apart. It will be appreciated that the visual indicators 44 may be visible via the visualization tool 38 of the endoscope 36 if the endoscope 36 is positioned such that the visualization tool 38 is either proximal of the proximal region 18 of the implantable medical device 10, or is positioned within the inflow region 24 (shown in FIG. 1) of the implantable medical device 10.

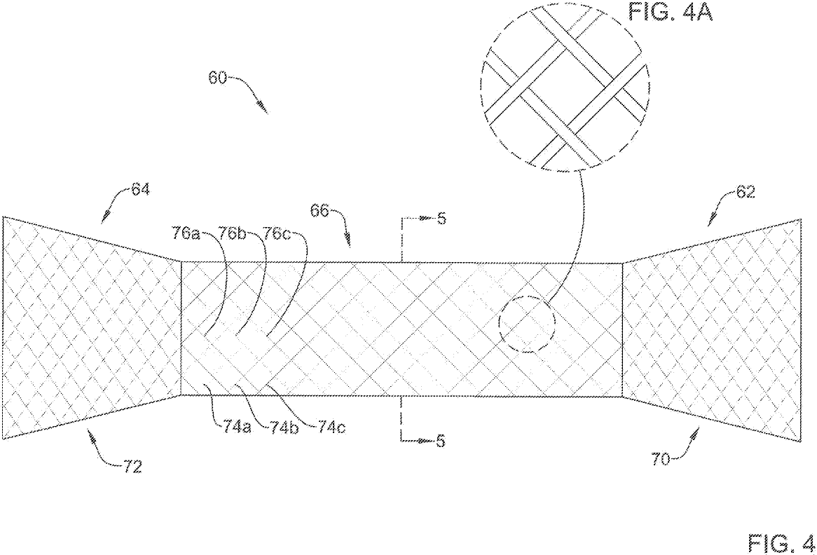

FIG. 4 is a side view of an illustrative endoluminal implant 60, such as, but not limited to, a stent. In some instances, the stent 60 may take the form of an elongated tubular member. While the stent 60 is described and shown as generally tubular, it is contemplated that the stent 10 may take any cross-sectional shape desired. The stent 60 may have a first, or proximal end 62, a second, or distal end 62 and an intermediate region 66 that is disposed between the first end 62 and the second end 64. The stent 60 may include a lumen 68 that extends from a first opening adjacent the first end 62 to a second opening adjacent the second end 64 to allow for the passage of food, fluids, etc. to pass therethrough.

The stent 60 may be expandable from a first radially collapsed configuration (not explicitly shown) to a second radially expanded configuration. In some cases, the stent 60 may be deployed to a configuration that is between the collapsed configuration and the expanded configuration, i.e., the stent 60 may be deployed having a deployed diameter that is greater than a diameter of the stent 60 or a particular portion thereof while in its collapsed configuration yet less than a diameter of the stent 60 or a particular portion thereof while in its fully expanded configuration. In some cases, the anatomy in which the stent 60 is deployed may influence its deployed configuration. For example, if the anatomy in which the stent 60 is to be deployed has a diameter that is less than a diameter of the stent 60 or a particular portion thereof when fully expanded, the stent 60 may have a deployed diameter that is intermediate its collapsed configuration diameter and its fully expanded configuration diameter.

The stent 60 may be formed of a plurality of interwoven filaments forming a tubular scaffold. For example, the tubular scaffold of the stent 60 may have a braided structure, fabricated from a plurality of filaments including a first plurality of filaments that each extend in a first helical direction and a second plurality of filaments that each extend in a second helical direction. As seen in FIG. 4A, the filaments are woven together such that a filament passes over a first filament and then passes under a second filament, for example. The tubular scaffold may include cells or interstices formed between adjacent filaments extending generally parallel in the first helical direction which intersect with adjacent filaments extending generally parallel in the second helical direction. It is contemplated that the filament(s) of the stent 60 may be made from a number of different materials such as, but not limited to, metals, metal alloys, shape memory alloys and/or polymers, as desired, enabling the stent 60 to be expanded into shape when accurately positioned within the anatomy. In some instances, the material may be selected to enable the stent 60 to be removed with relative ease as well. For example, the stent 60 may be formed from alloys such as, but not limited to, Nitinol and Elgiloy^{®}.

Depending on the material selected for construction of the stent 60, the stent 60 may be self-expanding, i.e., configured to automatically radially expand when unconstrained. In some instances, the stent 60 may not be self-expanding, and thus may not regain its fully expanded configuration without the assistance of an expansion device such as but not limited to an inflatable balloon disposed within the lumen 68. As used herein, the term “self-expanding” refers to the tendency of the stent 60 to return to a preprogrammed diameter when unrestrained by an external biasing force, e.g., a delivery catheter or sheath. While not shown, the stent 60 may include a one-way valve, such as an elastomeric slit valve or duck bill valve, positioned within the lumen 18 in order to prevent retrograde flow of gastrointestinal fluids, for example.

In some instances, in the radially expanded configuration, the stent 60 may include a first end region 70 proximate the first end 62 and a second end region 72 proximate the second end 74. In some cases, as illustrated, the first end region 70 and the second end region 72 may include retention features or anti-migration flared regions having enlarged outer diameters relative to the outer diameter of the intermediate region 66. The anti-migration flared regions, which may be positioned adjacent to the first end 62 and/or the second end 64, may be configured to engage an interior portion of the walls of the esophagus or other body lumen, for example. In some cases, the retention features, or flared regions, may have a larger outer diameter than the intermediate region 66 of the stent 60 in order to prevent the stent 60 from migrating once placed in the esophagus or other body lumen. In some instances, a transition from the cross-sectional area of the intermediate region 66 to the retention features or flared regions may be gradual, sloped, or occur in an abrupt step-wise manner, as desired.

In some instances, the first anti-migration flared region may have a first outermost diameter and the second anti-migration flared region may have a second outermost diameter. In some instances, the first and second outermost diameters may be approximately the same, while in other instances, the first and second outermost diameters may be different. In some cases, the stent 60 may include only one or none of the anti-migration flared regions. For example, the first end region 70 may include an anti-migration flare while the second end region 72 may have an outer diameter similar to the intermediate region 66. It is further contemplated that the second end region 72 may include an anti-migration flare while the first end region 70 may have an outer diameter similar to an outer diameter of the intermediate region 66. In some embodiments, the stent 60 may have a uniform outer diameter from the first end 62 to the second end 64. In some embodiments, the outer diameter of the intermediate region 66 may be in the range of 15 to 25 millimeters in the fully expanded configuration. The outer diameter of the anti-migration flares may be in the range of 20 to 30 millimeters in the fully expanded configuration. It is contemplated that the outer diameter of the stent 60 may be varied to suit the desired application.

In some cases, the stent 60 includes a first plurality of filaments that extend in a first helical direction and a second plurality of filaments that extend in a second helical direction. The stent 60 includes, for example, individual filaments 74a, 74b and 74c, each extending in a first helical direction. The stent 60 includes additional filaments (not referenced) extending in the first helical direction. The first helical direction may be considered as extending left to right, or proximal to distal, in a clockwise direction. The stent 60 includes, for example, individual filaments 76a, 76b and 76c, each extending in a second helical direction. The stent 60 includes additional filaments (not referenced) extending in the second helical direction. The second helical direction may be considered as extending left to right, or proximal to distal, in a counter-clockwise direction.

The stent 60 may be implanted in any desired fashion. This may include implanting the stent 60 in a blood vessel, esophagus, stomach, trachea, colon, intestine or other body lumen using a stent delivery device that, in the case of the stent 60 being self-expanding, may include an elongate shaft having an outer sheath that is configured to constrain the stent 60 in a collapsed delivery configuration until the outer sheath is withdrawn to allow the stent 60 to regain its expanded deployment configuration. In some cases, the stent 60 may be pushed out of the lumen of the elongate shaft of the stent delivery device in order to deploy the stent 60. If the stent 60 is not self-expanding, the stent delivery device may include a balloon, and expandable frame, or similar structure to expand the stent 60 from its collapsed delivery configuration to its expanded deployment configuration. In some cases, the stent 60 may be delivered endoscopically.

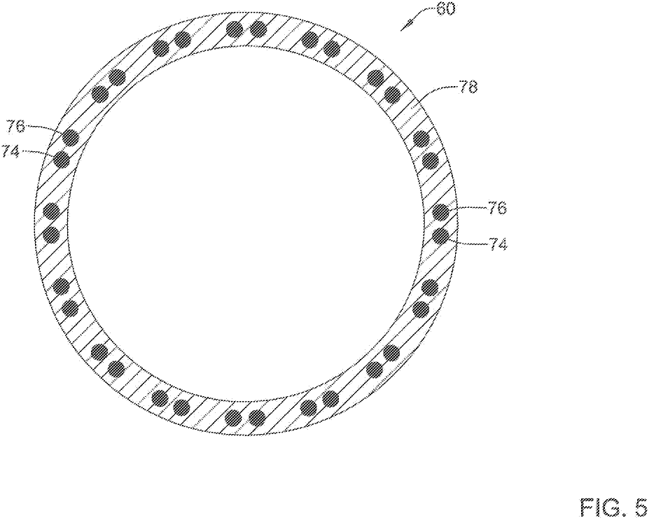

FIG. 5 is a cross-sectional view taken along line 5-5 of FIG. 4, showing the stent 60. As seen, the stent 60 includes a polymeric layer 78 with a number of filaments 74, 76 that are disposed within the polymeric layer 78. In some cases, the filaments 74, 76 may be considered as representing the filaments 74a, 74b and 74c extending in the first helical direction as well as the filaments 76a, 76b and 76c extending in the second helical direction. The stent 60 may include a one-over one-under pattern, with each filament 74 extending in the first helical direction alternately crossing over and under the filaments 76 extending in the second helical direction and each filament 76 extending in the second helical direction alternately crossing over and under the filaments 74 extending in the first helical direction. Alternatively, the stent 60 may include a two-over two-under pattern, or another pattern, as desired.

As discussed with respect to the implantable medical device 10 shown in FIGS. 1 through 3, there may a desire to be able to visualize the position or orientation of the stent 60 once the stent 60 has been implanted, regardless of how the stent 60 is implanted. Accordingly, the stent 60 may include visual indicators that may be visible from within or near the stent 60 by viewing along the central longitudinal axis of the stent 60 after the stent 60 has been implanted in the body lumen, such as endoscopically viewing an interior of the stent 60.

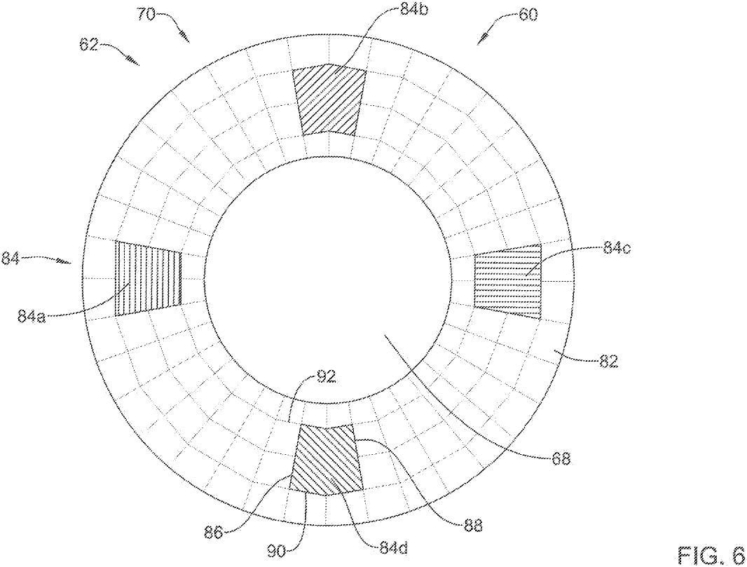

FIG. 6 is an end view of the stent 60 looking along the central longitudinal axis of the stent 60, showing the first or proximal end 62 and the accompanying first end region 70. It will be appreciated that the first or proximal end 62 of the stent 60 would be endoscopically visible using an endoscope or similar tool equipped with a lens or camera (such as the visualization tool 38 shown in FIG. 1) that was positioned within the body lumen into which the stent 60 was implanted. Looking at FIG. 6, the lumen 68 of the stent 60 can be seen, surrounded by an interior surface 82 of a flared portion of the stent 60 that can visually be seen with the visualization tool of an endoscope. The interior surface 82 corresponds to an inner surface of the flared anti-migration feature within the first end region 70.

As seen in FIG. 6, the stent 60 includes a total of four visual indicators 84, individually labeled as 84a, 84b, 84c and 84d. As shown, each of the four visual indicators 84 occupy a total of four cells (e.g., a 2 × 2 grid of cells) of the braided structure forming the stent 60. Looking, for example, at the visual indicator 84d, the visual indicator 44d occupies the four-cell region formed by the intersection of filaments 86, 88, 90 and 92. In some cases, at least some of the visual indicators 84 may be larger than a four cell-region. At least some of the visual indicators 84 may be smaller than a four-cell region. In some cases, each of the visual indicators 84 may be a different size in order to make it easier to visually distinguish between a particular visual indicator 84 and the other visual indicators 84. It will be appreciated that once the stent 60 has been implanted, the visual indicators 84 may remain visible via the visualization tool 38 of the endoscope 36 by placing the endoscope 36 such that the visualization tool 38 is either proximal of the stent 60, or such that the visualization tool 38 extends partway into the flared region of the stent 60, near the interior surface 82 of the stent 60. The visual indicators 84 are visible by viewing down the central longitudinal axis of the stent 60 and/or from a position within the stent 60, and may not be visible from a position radially outwardly from the stent 60 as the outer surfaces of the stent 60 may be in contact with tissue.

In some cases, each of the visual indicators 84 may be a unique color in order to more easily distinguish between the visual indicators 84. Some unique colors that could be used include, black, white, red, blue, green, yellow, orange, purple, etc. As an illustrative but non-limiting example, say that the visual indicator 84a is white, the visual indicator 84b is blue, the visual indicator 84c is yellow and the visual indicator 84d is red. As another example, say that the visual indicator 84a is green, the visual indicator 84b is orange, the visual indicator 84c is purple and the visual indicator 84d is yellow. Depending on the light wavelengths at which the visualization tool 38 is most receptive to, other colors may be used as well. The visual indicators 84 may be formed in any fashion. As an example, each of the visual indicators 84 may be formed by adding a particular pigment or other source of color to the polymeric layer 78, or a pigmented polymeric material applied to the polymeric layer 78.

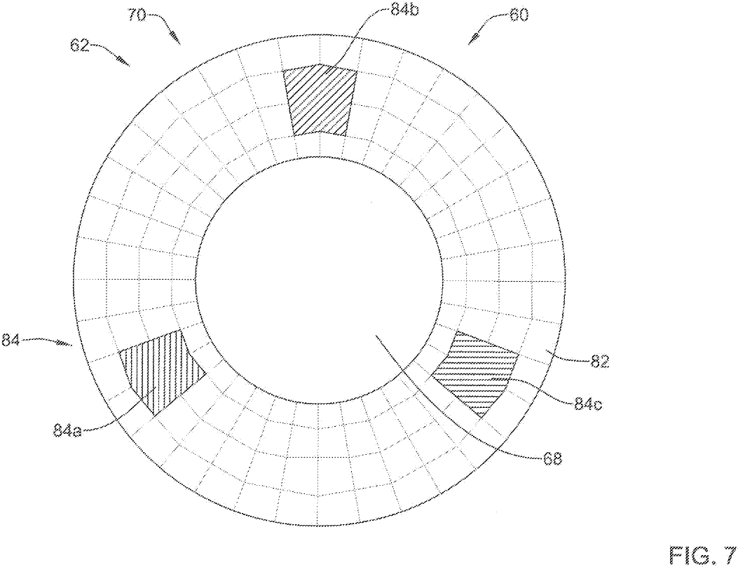

In FIG. 6, the stent 60 includes a total of four visual indicators 84 that are circumferentially equidistantly spaced about 90 degrees apart around the central longitudinal axis of the stent 60. In some cases, as seen for example in FIG. 7, the stent 60 may include a total of three visual indicators 84, individually labeled as 84a, 84b and 84c, that are circumferentially equidistantly spaced about 120 degrees apart around the central longitudinal axis of the stent 60. In some cases, at least some of the visual indicators may be separately formed and subsequently secured in place to the interior surface 82. For example, colored discs or other shapes may be adhesively secured to the interior surface 82. If the colored discs are metallic, they may be welded or soldered into position on the interior surface 82, such as by welding or soldering the colored discs to one or more of the metallic filaments forming the tubular scaffold of the stent 60. In some cases, the visual indicators may be considered as being equidistantly spaced circumferentially around the central longitudinal axis of the stent 60. It will be appreciated that the visual indicators 84 may remain visible via the visualization tool 38 of the endoscope 36 by positioning the endoscope 36 such that the visualization tool 38 is either proximal of the stent 60, or such that the visualization tool 38 extends partway into the flared region of the stent 60, near the interior surface 82 of the stent 60. The visual indicators 84 are visible by viewing down the central longitudinal axis of the stent 60 and/or from a position within the stent 60, and may not be visible from a position radially outwardly from the stent 60 as the outer surfaces of the stent 60 may be in contact with tissue.

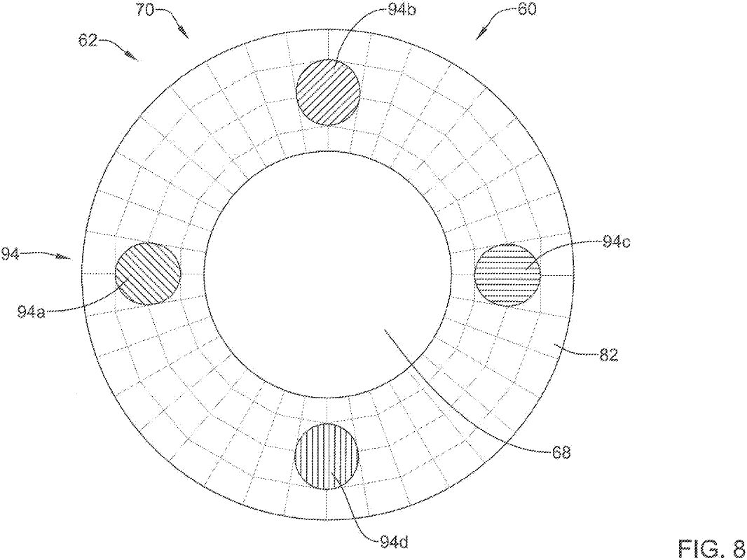

FIG. 8 is an end view of the stent 60, showing a total of four visual indicators 94, individually labeled as 94a, 94b, 94c and 94d. As shown, each of the visual indicators 94 are circular. However, in some instances, each visual indicator 94 may be a unique color, a unique shape and/or a unique size relative to the other visual indicators 94. As an illustrative but non-limiting example, the visual indicator 94a may be a circle, the visual indicator 94b may be a square, the visual indicator 94c may be a triangle, and the visual indicator 94d may be a polygon having at least five sides. As another illustrative but non-limiting example, the visual indicator 94a may be a circle having a diameter of 1 millimeter (mm), the visual indicator 94b may be a circle having a diameter of 2 mm, the visual indicator 94c may be a circle having a diameter of 3 mm and the visual indicator 94d may be a circle having a diameter of 4 mm. These are just examples.

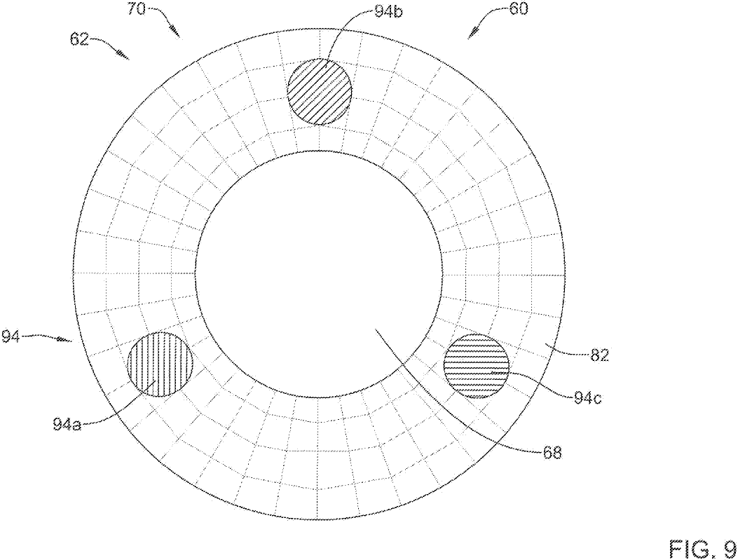

In FIG. 8, the stent 60 includes a total of four visual indicators 94 that are circumferentially equidistantly spaced about 90 degrees apart around the central longitudinal axis of the stent 60. In some cases, as seen for example in FIG. 9, the stent 60 may include a total of three visual indicators 94, individually labeled as 84a, 84b and 84c, that are circumferentially equidistantly spaced about 120 degrees apart around the central longitudinal axis of the stent 60. In some cases, at least some of the visual indicators may be separately formed and subsequently secured in place to the interior surface 82. It will be appreciated that the visual indicators 94 may remain visible via the visualization tool 38 of the endoscope 36 by positioning the endoscope 36 such that the visualization tool 38 is either proximal of the stent 60, or such that the visualization tool 38 extends partway into the flared region of the stent 60, near the interior surface 82 of the stent 60. The visual indicators 94 are visible by viewing down the central longitudinal axis of the stent 60 and/or from a position within the stent 60, and may not be visible from a position radially outwardly from the stent 60 as the outer surfaces of the stent 60 may be in contact with tissue.

The devices described herein, and various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. An implantable stent, comprising:
an elongate body extending from a distal region to a proximal region, the elongate body including one or more filaments that are woven together to form the elongate body, the elongate body defining an outer stent surface and an inner stent surface; and
one or more visual indicators positioned relative to the inner stent surface such that the one or more visual indicators are visible endoscopically from a position within or proximate the implantable stent.

2. The implantable stent of claim 1, wherein the distal region or the proximal region includes a flared end, the inner stent surface extending into the flared end, and the one or more visual indicators are positioned relative to the inner stent surface within the flared end.

3. The implantable stent of claim 1, wherein the one or more visual indicators comprise two or more visual indicators that are circumferentially equidistantly spaced apart on the inner stent surface, each of the two or more visual indicators is visually distinguishable from the other position indicators.

4. The implantable stent of claim 3, wherein at least some of the two or more visual indicators comprise objects that are secured relative to the inner stent surface.

5. The implantable stent of claim 3, further comprising a polymeric coating over at least part of the elongate body.

6. The implantable stent of claim 5, wherein at least some of the two or more visual indicators comprise color changes introduced into the polymeric coating.

7. The implantable stent of claim 1, wherein at least one of the one or more visual indicators comprise a radiopaque material.

8. An implantable medical device adapted to be deployed endoscopically, the implantable medical device comprising:
an elongate body extending from a distal region to a proximal region, at least one of the distal region and the proximal region including a flared region defining an inner flared surface; and
two or more visual indicators that are secured relative to the inner flared surface, each of the two or more visual indicators positioned to be visible endoscopically.

9. The implantable medical device of claim 8, wherein each of the two or more visual indicators are visually distinguishable from others of the two or more visual indicators.

10. The implantable medical device of claim 8, wherein each of the two or more visual indicators comprise a color different from a color of any other of the two or more visual indicators.

11. The implantable medical device of claim 8, comprising three visual indicators that are equidistantly spaced apart on the inner flared surface.

12. The implantable medical device of claim 8, comprising four visual indicators that are equidistantly spaced apart on the inner flared surface.

13. The implantable medical device of claim 8, wherein at least some of the two or more visual indicators comprise objects that are secured relative to the inner flared surface.

14. The implantable medical device of claim 8, further comprising a polymeric coating over at least part of the elongate body.

15. The implantable medical device of claim 8, wherein at least some of the two or more visual indicators comprise color changes introduced into the polymeric coating.

16. The implantable medical device of claim 8, wherein at least one of the two or more visual indicators comprise a radiopaque material.

17. An implantable stent, comprising:
an elongate body extending from a distal region to a proximal region, the elongate body including one or more filaments that are woven together to form the elongate body, the elongate body moveable between a collapsed configuration for delivery and an expanded configuration for deployment, the elongate body including a constant diameter main segment and a flared end segment, the flared end segment including an inner flared surface and an outer flared surface; and
a plurality of visual indicators that are secured relative to the inner flared surface such that each of the plurality of visual indicators are visible endoscopically.

18. The implantable stent of claim 17, wherein the plurality of visual indicators comprise three visual indicators that are equidistantly spaced apart on the inner stent surface, each of the three visual indicators a unique color relative to the other two visual indicators.

19. The implantable stent of claim 17, wherein the plurality of visual indicators comprise four visual indicators that are equidistantly spaced apart on the inner stent surface, each of the four visual indicators a unique color relative to the other three visual indicators.

20. The implantable stent of claim 17, wherein at least some of the plurality of visual indicators comprise a pigment added to a portion of a polymeric coating disposed over the elongate body.
